(19) United States
(12) Patent Application Publication
(10) Pub. No.: US 2012/0231514 A1
(43) Pub. Date: Sep. 13, 2012

(54) FERMENTATION METHOD TO PRODUCE A LIGNOCELLULOSE-BASED SUGAR STREAM WITH ENRICHED PENTOSE CONTENT

(75) Inventors: Jan-Maarten A. Geertman, Voorschoten (NL); Azher Razvi, Gloucester (CA)

(73) Assignee: Iogen Energy Corporation, Ottawa, ON (CA)

(21) Appl. No.: 13/392,997

(22) PCT Filed: Aug. 30, 2010
(86) PCT No.: PCT/CA10/01342

§ 371 Date: May 7, 2012

Related U.S. Application Data
(60) Provisional application No. 61/238,446, filed on Aug. 31, 2009.

(51) Int. Cl. C12P 7/18

(52) U.S. Cl. 435/158

(57) ABSTRACT

A method for producing a fermented solution enriched in xylose is provided. The sugar stream that is fermented results from the hydrolysis of a lignocellulosic feedstock and comprises at least a hexose and a pentose. The method comprises fermenting the sugar stream resulting from the hydrolysis of the lignocellulosic feedstock with a microorganism that preferentially ferments the hexose over the pentose. The fermenting is conducted under aerobic conditions and comprises continuously feeding the sugar stream to a fermentation reactor at a dilution rate that converts the hexose to cell mass preferentially over the pentose, thereby reducing the concentration of the glucose in the sugar stream so that the fermented solution thus produced is enriched in the pentose relative to said sugar stream.

### FIELD OF INVENTION

The present invention relates to a method involving fermenting a sugar stream originating from a lignocellulosic feedstock. More specifically, the present invention provides a method for producing a fermented solution enriched in a pentose sugar from a sugar stream resulting from the hydrolysis of a lignocellulosic feedstock, the sugar stream comprising a hexose sugar and a pentose sugar.

### BACKGROUND OF THE INVENTION

In recent years there has been an increasing interest in generating ethanol and fine chemicals from lignocellulosic feedstocks. These feedstocks are of particular interest as they are inexpensive and are often burned or landfilled. Accordingly, there is an enormous untapped potential for their use as a source of fermentable sugar to produce ethanol or other byproducts. A fermentable sugar solution is produced from the polysaccharide components of the feedstock, namely cellulose and hemicellulose. Cellulose makes up 30% to 50% of most of the key feedstocks, while hemicellulose is present at 15% to 30% in most feedstocks.

In order to produce sugar from lignocellulosic feedstocks, it is first necessary to break them down into their composite sugar molecules. This can be accomplished by physical and/or chemical pretreatment. Examples of chemical pretreatment are acid pretreatment (see U.S. Pat. No. 4,461,648) or alkali pretreatment, such as Ammonia Fiber Explosion (AFEX) pretreatment. Acid pretreatment hydrolyzes most of the hemicellulose, but there is little conversion of the cellulose to glucose. On the other hand, alkali pretreatment methods may or may not hydrolyze hemicellulose, although in either case the base reacts with acidic groups present on the hemicellulose to open up the surface of the substrate. After pretreatment with acid or alkali, the cellulose may then be hydrolyzed to glucose by cellulase enzymes or by further chemical treatment. Glucose can then be fermented to fuels including, but not limited to, ethanol or butanol or chemicals, examples of which include sugar alcohols and organic acids.

It is also known to hydrolyze the lignocellulosic feedstock in a single step with acid or alkali. This involves employing harsher conditions to effect hydrolysis of both the hemicellulose and cellulose components of the feedstock.

Hydrolysis of the hemicellulose component of lignocellulosic feedstocks yields a mixture of pentose and hexose sugars, namely xylose, galactose, mannose and arabinose. The pentose sugars, xylose and arabinose, can be fermented to ethanol by recombinant yeast (see U.S. Pat. No. 5,789,210 (Ho et al.), U.S. Pat. No. 5,126,266 (Jeffries et al.), WO 2008/130603 (Abbas et al.) and WO 03/095627 (Boles and Becker)) or by bacteria. Alternatively, the pentose sugars may be used as starting materials for the generation of other high value products using chemical or biological processes. For example, in recent years, the production of xylitol from xylose has received much attention because of its value as a substitute sugar sweetener. Advantages of using xylitol over sucrose as a sweetener are that it contains fewer calories per gram and has been reported to reduce tooth decay. Xylitol can be produced fermentatively from xylose by yeasts such as Candida, or by chemical hydrogenation using a metal catalyst.

When converting a sugar to a commercial product, it is desirable, in some circumstances, that the sugar solution fed to the process does not contain substantial quantities of unwanted sugars. For example, during the conversion of xylose to xylitol from a hemicellulose hydrolyzate, other sugars present in the feed are converted to their corresponding sugar alcohols, thus necessitating separation steps to remove them. This can add significant cost to the process.

U.S. Pat. No. 5,081,026 discloses a process for producing xylitol from a hemicellulose hydrolyzate containing xylose as well as hexose impurities. According to the process, xylose is fermented to xylitol by Candida yeast and the hexose impurities are converted to ethanol. The small amounts of ethanol produced from the unwanted sugars can then be evaporated to separate the xylitol from ethanol. However, the process requires an evaporation unit to drive off the ethanol, which adds to the cost and complexity of the process.

A further problem arising from the fermentation of xylose or other pentose sugars originating from lignocellulosic feedstocks is the presence of fermentation inhibitors in sugar-containing streams resulting from hydrolysis of the feedstock. For example, after pretreatment of a lignocellulosic feedstock with acid, the resulting aqueous hydrolyzate stream will contain acetic acid originating from acetyl groups present on the hemicellulose and lignin components of the feedstock. The presence of acetic acid in lignocellulose hydrolyzates is especially problematic as it inhibits yeast cell growth and thus can significantly reduce the yield of fermentation products (Abbott et al., 2007, FEMS Yeast Res. 7:819-833). Other yeast inhibitors that arise when converting lignocellulosic feedstocks to fermentable sugars are furfural and 5-hydroxymethylfurfural (HMF). Furfural and HMF result from the loss of water molecules from xylose and glucose, respectively, by exposure to high temperatures and acid. The inhibitory effects of these compounds decrease the efficiency of the fermentation operations by lengthening the time required for carrying out the fermentation, increasing the amount of yeast required, decreasing the final yields, or a combination of these.

One method that has been proposed to reduce the concentration of inhibitors arising from hydrolysis of lignocellulosic feedstocks is overliming which involves the addition of Ca(OH)₂ to precipitate inhibitors from lignocellulosic hydrolysates, thereby improving the fermentation. Such processes are disclosed by U.S. Pat. Nos. 2,203,360, 4,342,831, 6,737,258, 7,455,997 and Wooley et al. (In Lignocellulosic Biomass to Ethanol Process Design and Economics Utilizing Co-Current Dilute Acid Prehydrolysis and Enzyme Hydrolysis Current and Future Scenarios, (1999) Technical Report, National Renewable Energy Laboratory pp. 16-17). However, any handling of the lime cake is difficult and costly. In addition, the introduction of calcium into the stream increases the likelihood that calcium scale will deposit on evaporators, distillation columns, and other process equipment. The clean-up and avoidance of scale increases the cost of sugar processing.

Another method that has been proposed to remove inhibitors of fermentation is ion exchange. For example, ion exchange has been investigated by Nilvebrant et al. (App. Biochem. Biotech., 2001, 91-93:35-49) in which a spruce hydrolyzate was treated to remove fermentation inhibitors, such as phenolic compounds, furan aldehydes and aliphatic acids. U.S. Pat. No. 7,455,997 and Wooley et al. (supra) report the use of ion exchange to remove acetic acid from an acid hydrolyzed mixture obtained from wood chips, followed by lime treatment. Similarly, Watson et al. (Enzyme Microb. Technol., 1984, 6:451-456) disclose the use of ion exchange to remove inhibitors, such as acetic acid and 2-furaldehyde (furfural), from a sugar can bagasse acid hydrolyzate prior to fermentation. Furthermore, Tran and Chambers (Enzyme Microb. Technol., 1986, 8:439-444) disclose various treatments to remove inhibitors prior to fermentation from an acid prehydrolysate from red oak, including mixed bed ion resin treatment.

In practice, several factors limit the effectiveness of ion exchange treatment to remove inhibitors. First, the multi-component nature of the streams results in an inefficient removal of some species at any single set of conditions. Second, the high ionic load demands very frequent and expensive regeneration of the resin. Finally, not all of the inhibitors are ionic, and ion exchange is ineffective in removing nonionic compounds from sugar.

US 2008/0171370 reports that gallic acid can be used to detoxify hydrolyzates resulting from pretreating a lignocellulosic material by binding acetic acid. As disclosed therein, the gallic acid is a natural polymer co-monomer, i.e., the core of the gallotannin structure, and therefore is a natural means to polymerize phenols and acetic acid in a Fischer esterification with a sulphuric acid catalyst.

WO 2008/124162 discloses the selective removal of acetate from a sugar mixture containing xylose and glucose by an E. coli strain that is able to convert acetate to a biochemical such as ethanol, butanol, succinate, lactate, fumarate, pyruvate, butyric acid and acetone. The E. coli has been deleted in four genes that would otherwise code for proteins involved in xylose and glucose utilization, thereby preventing the consumption of either xylose or glucose by the E. coli, but that have no known effect on acetate metabolism. After acetate conversion to the biochemical, xylose and glucose fermentation are conducted on the sugar mixture using separate microorganisms, one with the ability to only ferment xylose, and the other with the ability to only ferment glucose. However, the process is not directed to removing unwanted sugars from a sugar hydrolyzate, but rather to maximizing the conversion of all sugars present in the mixture to ethanol or other biochemicals.

### SUMMARY OF THE INVENTION

The present invention relates to a method involving fermenting a sugar stream originating from a lignocellulosic feedstock. More specifically, the present invention provides a method for producing a fermented solution enriched in a pentose sugar from a sugar stream resulting from the hydrolysis of a lignocellulosic feedstock, the sugar stream comprising at least a hexose sugar and a pentose sugar.

It is an object of the invention to provide a method for producing a fermented solution that is enriched in a pentose sugar.

The present invention provides a process for conducting a preliminary fermentation of a sugar stream (referred to herein as a “first-stage fermentation”) obtained from a lignocellulosic feedstock in order to produce a solution comprising a pentose sugar and that contains reduced levels of a hexose sugar. Specifically, the process involves fermenting the sugar stream with a microorganism in the first-stage fermentation under conditions that result in the conversion of at least the hexose to cell mass by the microorganism. A benefit of the process is that it results in the production of cell mass from a sugar component that otherwise could have a deleterious effect on a later fermentation in which the pentose is converted to a fermentation product (referred to herein as a “second-stage fermentation”) or a chemical compound produced by a non-biological conversion process. Moreover, the process of the invention can reduce the requirements for producing microorganisms for the fermentation by recycle or by other means.

Furthermore, in embodiments of the invention, acetate is converted to cell mass by the microorganism utilized in the first-stage fermentation along with the glucose. Advantageously, by reducing the concentration of acetate, and thus its inhibitory effect on the microorganism, the xylose can be converted in the subsequent fermentation to a higher value product(s) with improved efficiency.

Thus, according to a first aspect of the invention, there is provided a method for producing a fermentation product from xylose, the method comprising:
(i) obtaining a sugar stream comprising at least glucose and xylose, the sugar stream resulting from the hydrolysis of a lignocellulosic feedstock;
(ii) reducing the concentration of glucose in the sugar stream by fermenting the sugar stream in a first-stage fermentation with a yeast that preferentially ferments the glucose over the xylose, the fermenting being conducted under aerobic conditions and comprising continuously feeding the sugar stream to a fermentation reactor at a dilution rate that allows the yeast to produce cell mass from the glucose preferentially over the xylose so as to produce a fermented solution that is enriched in the xylose relative to the sugar stream; and
(iii) subsequent to step (ii), fermenting the xylose remaining after the first-stage fermentation with yeast to produce the fermentation product.

According to an embodiment of the invention, the fermented solution produced in the first-stage fermentation has a xylose:glucose ratio of at least 20:1 (wt:wt).

The sugar stream may further comprise acetate and wherein the acetate is converted to cell mass in the first-stage fermentation by the microorganism.

In a further embodiment of the invention, the yeast in the first and second stage fermentations is identical.

According to an embodiment of the invention, the first-stage fermentation is a continuous process or a fed-batch process.

The sugar stream resulting from the hydrolysis of the lignocellulosic feedstock may further comprise furfural. The first-stage fermentation may be conducted so that the furfural is converted to furoic acid or furfural alcohol by the yeast utilized in the first-stage fermentation. Moreover, the sugar stream resulting from the hydrolysis of the lignocellulosic feedstock may further comprise 5-hydroxymethylfurfural. According to this embodiment, the first-stage fermentation can be conducted so that the 5-hydroxymethylfurfural is converted to a less inhibitory analog by the yeast utilized in the first-stage fermentation.

According to another embodiment, the xylose makes up at least about 65 wt % of the combined glucose and xylose content of the sugar stream.

In yet another embodiment of the invention, the sugar stream is produced by pretreating the lignocellulosic feedstock with acid or alkali so as to produce a composition comprising pretreated feedstock and then separating the sugar stream from said composition. Alternatively, the sugar stream results from pretreating a lignocellulosic feedstock with acid or alkali to produce a composition comprising pretreated feedstock, followed by hydrolyzing cellulose present in said pretreated feedstock.

According to a further embodiment of the invention, the yeast in the first-stage fermentation and the second-stage fermentation is a yeast strain that ferments the xylose to xylitol. According to this embodiment, the yeast strain may belong to a genus selected from the group consisting of Candida, Pichia, Pachvsolen, Hansenula, Debaryomyces, Kluyveromyces and Schizosaccharomyces. In one embodiment of the invention, the yeast is Candida, including, but not limited to, Candida tropicalis.

According to a second aspect of the invention, there is provided a method for producing xylitol from xylose, said method comprising:
(i) obtaining a sugar stream comprising at least glucose and xylose, said sugar stream resulting from chemical pretreatment of a lignocellulosic feedstock, wherein the sugar stream contains at least 65% xylose relative to the combined content of glucose and xylose present in said sugar stream;
(ii) reducing the concentration of glucose and acetate in the sugar stream by fermenting said sugar stream in a first-stage fermentation with a Candida, Pichia, Pachysolen, Hansenula, Debaryomyces, Kluyveromyces or Schizosaccharomyces strain that preferentially ferments the glucose over the xylose, said fermenting being conducted under aerobic conditions and comprising continuously feeding the sugar stream to a fermentation reactor at a dilution rate that allows the yeast to produce cell mass from the glucose preferentially over the xylose so as to produce a fermented solution that is enriched in the xylose relative to said sugar stream; and
(iii) subsequent to step (ii), fermenting the xylose in the fermented solution with the Candida, Pichia, Pachvsolen, Hansenula, Debaryomyces, Kluyveromyces or Schizosaccharomyces strain to produce the xylitol. In one non-limiting example, the Candida strain is Candida tropicalis.

According to a third aspect of the invention, there is provided a method for producing a fermentation product from xylose, said method comprising:
(i) obtaining a sugar stream comprising at least glucose and xylose, said sugar stream resulting from the hydrolysis of a lignocellulosic feedstock;
(ii) reducing the concentration of glucose in the sugar stream by fermenting said sugar stream in a first-stage fermentation with a microorganism that preferentially ferments the glucose over the xylose, said fermenting being conducted under aerobic conditions and comprising continuously feeding the sugar stream to a fermentation reactor at a dilution rate that allows the yeast to produce cell mass from the glucose preferentially over the xylose so as to produce a fermented solution that is enriched in the xylose relative to said sugar stream; and
(iii) subsequent to step (ii), fermenting the xylose remaining after the first-stage fermentation with a microorganism to produce the fermentation product.

The invention also encompasses conducting the first-stage fermentation on a sugar stream comprising a hexose sugar selected from the group consisting of glucose, galactose, rhamnose, fructose and mannose, and a combination thereof and a pentose sugar selected from the group consisting of xylose, arabinose and fucose, and a combination thereof. That is, although glucose conversion to cell mass in the first-stage fermentation is specifically described herein, as well as the conversion of xylose to a fermentation product in a second-stage fermentation, the principle of the invention is applicable to the conversion of any hexose sugar to cell mass in the first-stage fermentation and the subsequent conversion of any pentose sugar remaining after the first-stage fermentation to a chemical compound of interest by fermentation or non-biological means.

Thus, according to a fourth aspect of the invention, there is provided a method for producing a fermentation product from a pentose sugar, said method comprising:
(i) obtaining a sugar stream comprising at least a hexose sugar and a pentose sugar, said sugar stream resulting from the hydrolysis of a lignocellulosic feedstock;
(ii) fermenting said sugar stream resulting from the hydrolysis of the lignocellulosic feedstock with a microorganism that preferentially ferments the hexose sugar over the pentose sugar, said hexose sugar selected from the group consisting of glucose, galactose and mannose and said pentose sugar selected from the group consisting of xylose, arabinose and fucose, said fermenting being conducted under aerobic conditions and comprising continuously feeding the sugar stream to a fermentation reactor at a dilution rate that converts the hexose sugar to cell mass preferentially over the pentose sugar, thereby reducing the concentration of the hexose sugar in the sugar stream so that the fermented solution thus produced is enriched in the pentose sugar relative to said sugar stream; and
(iii) subsequent to step (ii), converting the pentose sugar to a chemical compound.

In one embodiment of the fourth aspect of the invention, the sugar stream resulting from the hydrolysis of the lignocellulosic feedstock further comprises furfural and wherein the furfural is converted to furoic acid or furfural alcohol by the microorganism in the first-stage fermentation.

According to a further embodiment of the invention, the sugar stream resulting from the hydrolysis of the lignocellulosic feedstock further comprises 5-hydroxymethylfurfural and wherein the 5-hydroxymethylfurfural is converted to a less inhibitory analog by the microorganism in the first-stage fermentation.

According to another embodiment of the fourth aspect of the invention, the sugar stream is produced by pretreating the lignocellulosic feedstock with acid or alkali to produce a composition comprising pretreated feedstock and then separating the sugar stream from said composition.

According to another embodiment of the fourth aspect of the invention, the sugar stream results from pretreating a lignocellulosic feedstock with acid or alkali to produce a composition comprising pretreated feedstock, followed by hydrolyzing cellulose present in said pretreated feedstock.

According to any one of the above aspects of the invention or embodiments of any of the aforementioned aspects, after the first-stage fermentation is complete, the microorganisms in the fermented solution can be separated from the solution. The resultant solution from which the cells are removed, and which is enriched in the pentose sugar, can be sold as a commercial product, typically after purification. Thus, the present invention also relates to producing a fermented solution enriched in a pentose sugar, which fermented solution results from any one of the first-stage fermentations set forth above. The pentose sugar may then be recovered from the fermented solution after removal of the cells. This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 is a schematic flow diagram depicting the production of a sugar stream that is fed to a first-stage fermentation according to one embodiment of the invention;

FIG. 2 is a schematic flow diagram depicting the production of a sugar stream that is fed to the first-stage fermentation according to another embodiment of the invention; and

FIG. 3 is a schematic depicting a two-stage fermentation to produce xylitol with Candida tropicalis according to an embodiment of the invention.

### DETAILED DESCRIPTION OF THE INVENTION

The following description is of an embodiment by way of example only and without limitation to the combination of features necessary for carrying the invention into effect.

Production of a Sugar Stream from Lignocellulosic Feedstocks

The sugar stream for use in the invention results from the hydrolysis of a ligmocellulosic feedstock. Representative lignocellulosic feedstocks are (1) agricultural wastes such as corn stover, corn cobs, wheat straw, barley straw, oat straw, rice straw, canola straw, and soybean stover; (2) grasses such as switch grass, miscanthus, cord grass, and reed canary grass; (3) forestry wastes such as aspen wood and sawdust; and (4) sugar processing residues such as bagasse and beet pulp. The feedstocks preferably contain high concentrations of cellulose and hemicellulose that are the source of the sugar in the aqueous stream.

Lignocellulosic feedstocks comprise cellulose in an amount greater than about 20%, more preferably greater than about 30%, more preferably greater than about 40% (wt/wt). For example, the lignocellulosic material may comprise from about 20% to about 50% (wt/wt) cellulose, or any amount therebetween. Hemicellulose may be present at 15% to 30% (wt/wt), or any amount therebetween. Furthermore, the lignocellulosic feedstock comprises lignin in an amount greater than about 10%, more typically in an amount greater than about 15% (wt/wt). The lignocellulosic feedstock may also comprise small amounts of sucrose, fructose and starch.

According to one embodiment of the invention, the sugar stream fed to the fermentation is a stream resulting from pretreating the feedstock with acid, i.e., a hemicellulose hydrolysate. The acid pretreatment is intended to deliver a sufficient combination of mechanical and chemical action so as to disrupt the fiber structure of the lignocellulosic feedstock and increase the surface area of the feedstock to make it accessible or more susceptible to cellulase enzymes. Preferably, the acid pretreatment is performed so that nearly complete hydrolysis of the hemicellulose and only a small amount of conversion of cellulose to glucose occurs. The majority of the cellulose is hydrolyzed to glucose in a subsequent step that uses cellulase enzymes, although a small amount of the cellulose can be hydrolyzed in the acid pretreatment step as well. Typically a dilute acid, at a concentration from about 0.02% (wt/wt) to about 5% (wt/wt), or any amount therebetween, (measured as the percentage weight of pure acid in the total weight of dry feedstock plus aqueous solution) is used for the pretreatment.

A preferred pretreatment, without intending to be limiting, is steam explosion described in U.S. Pat. No. 4,416,648 (Foody; which is incorporated herein by reference).

Examples of acids that can be used in the process include those selected from the group consisting of sulfuric acid, sulfurous acid, sulfur dioxide and a combination thereof. Preferably, the acid is sulfuric acid.

The acid pretreatment is preferably carried out at a maximum temperature of about 160° C. to about 280° C. The time that the feedstock is held at this temperature may be about 6 seconds to about 600 seconds. In one embodiment of the invention, the pH of the pretreatment is about 0.4 to about 3.0, or any pH range therebetween. For example, the pH of the pretreatment may be 0.4, 1.0, 1.5, 2.0, 2.5 or 3.0. Preferably, the pretreatment is carried out to minimize the degradation of xylose and the production of furfural.

In another embodiment of the invention, the chemical used for pretreatment of the lignocellulosic feedstock is alkali. The alkali used in the pretreatment reacts with acidic groups present on the hemicellulose to open up the surface of the substrate. With alkali pretreatment, acetate is produced from acetyl groups present on the hemicellulose component of the feedstock, although the amount of acetate present will vary depending on the severity of the treatment. However, in contrast to acid pretreatment, alkali pretreatment methods may or may not hydrolyze xylan to produce xylose.

Examples of alkali that may be used in the pretreatment include ammonia, ammonium hydroxide, potassium hydroxide, and sodium hydroxide. The pretreatment may also be conducted with alkali that is insoluble in water, such as lime and magnesium hydroxide, although the soluble bases are preferred.

An example of a suitable alkali pretreatment is Ammonia Freeze Explosion, Ammonia Fiber Explosion or Ammonia Fiber Expansion (“AFEX” process). According to this process, the lignocellulosic feedstock is contacted with ammonia or ammonium hydroxide in a pressure vessel for a sufficient time to enable the ammonia or ammonium hydroxide to alter the crystal structure of the cellulose fibers. The pressure is then rapidly reduced, which allows the ammonia to flash or boil and explode the cellulose fiber structure. (See U.S. Pat. Nos. 5,171,592, 5,037,663, 4,600,590, 6,106,888, 4,356,196, 5,939,544, 6,176,176, 5,037,663 and 5,171,592, which are each incorporated herein by reference). The flashed ammonia may then be recovered according to known processes.

Another suitable alkali pretreatment for use in the present invention employs dilute solutions of ammonia or ammonium hydroxide as set forth in US2009/0053770 and US2007/0031918, which are each incorporated herein by reference.

Yet a further non-limiting example of a pretreatment process for use in the present invention includes chemical treatment of the feedstock with organic solvents. Organic liquids in pretreatment systems are described by Converse et al., (U.S. Pat. No. 4,556,430; incorporated herein by reference) and such methods have the advantage that the low boiling point liquids can easily be recovered and reused. Other pretreatments, such as the Organosolv™ process, also use organic liquids (see U.S. Pat. No. 7,465,791, which is also incorporated herein by reference). Subjecting the feedstock to pressurized water may also be a suitable pretreatment method (See Weil, J. et al., 1997, Applied Biochemistry and Biotechnology, 68(1-2):21-40, which is incorporated herein by reference).

The pretreatment produces a pretreated feedstock composition (e.g., pretreated feedstock slurry) that contains a soluble component including the sugars resulting from hydrolysis of the hemicellulose, optionally acetic acid and other inhibitors, and solids including unhydrolyzed feedstock and lignin.

According to one embodiment of the invention, the soluble components of the pretreated feedstock composition are separated from the solids. The soluble fraction, which includes the sugars released during pretreatment and other soluble components, including inhibitors, may then be sent to the first-stage fermentation. It will be understood, however, that if the hemicellulose is not effectively hydrolyzed during the pretreatment, it may be desirable to include a further hydrolysis step or steps with enzymes or by further alkali or acid treatment to produce fermentable sugars.

The foregoing separation may be carried out by washing the pretreated feedstock composition with an aqueous solution to produce a wash stream, and a solids stream comprising the unhydrolyzed, pretreated feedstock. Alternatively, the soluble component is separated from the solids by subjecting the pretreated feedstock composition to a solids-liquid separation, using known methods such as centrifugation, microfiltration, plate and frame filtration, cross-flow filtration, pressure filtration, vacuum filtration and the like. Optionally, a washing step may be incorporated into the solids-liquids separation. The separated solids, which contain cellulose, may then be sent to enzymatic hydrolysis with cellulase enzymes in order to convert the cellulose to glucose. The resultant glucose-containing stream may then be fermented to ethanol, butanol or other fermentation products.

According to another embodiment of the invention, the pretreated feedstock composition is fed to the first-stage fermentation without separation of the solids contained therein. After the fermentation, the unhydrolyzed solids may be subjected to enzymatic hydrolysis with cellulase enzymes to convert the cellulose to glucose.

According to yet another embodiment of the invention, the pretreated feedstock composition, together with any sugars resulting from hemicellulose hydrolysis, is subjected to cellulose hydrolysis with cellulase enzymes and the resultant sugar stream is sent to the first-stage fermentation. A major component of the resulting sugar stream will be glucose, although xylose and other pentose sugars derived from the hemicellulose component will be present as well.

Prior to hydrolysis with cellulase enzymes, the pH of the pretreated feedstock slurry is adjusted to a value that is amenable to the cellulase enzymes, which is typically between about 4 and about 6, although the pH can be higher if alkalophilic cellulases are used.

The enzymatic hydrolysis can be carried out with any type of cellulase enzymes capable of hydrolyzing the cellulose to glucose, regardless of their source. Among the most widely studied, characterized and commercially produced cellulases are those obtained from fungi of the genera Aspergillus, Humicola, and Trichoderma, and from the bacteria of the genera Bacillus and Thermobifida. The cellulases typically comprise one or more CBHs, EGs and β-glucosidase enzymes and may additionally contain hemicellulases, esterases and swollenins. (See Lynd et al., 2002, Microbiology and Molecular Biology Reviews, 66(3):506-577 for a review of cellulase enzyme systems and Coutinho and Henrissat, 1999, “Carbohydrate-active enzymes: an integrated database approach.” In Recent Advances in Carbohydrate Bioengineering, Gilbert, Davies, Henrissat and Svensson eds., The Royal Society of Chemistry, Cambridge, pp. 3-12, each of which are incorporated herein by reference).

Following cellulose hydrolysis of the pretreated feedstock slurry, any insoluble solids, including, but not limited to lignin, present in the resulting sugar stream may be removed using conventional solid-liquid separation techniques prior to any further processing. These solids may be burned to provide energy for the entire process.

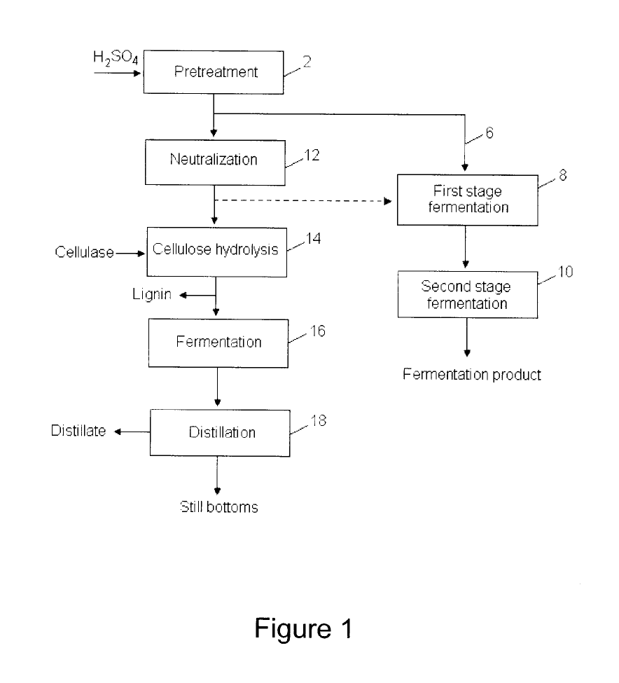

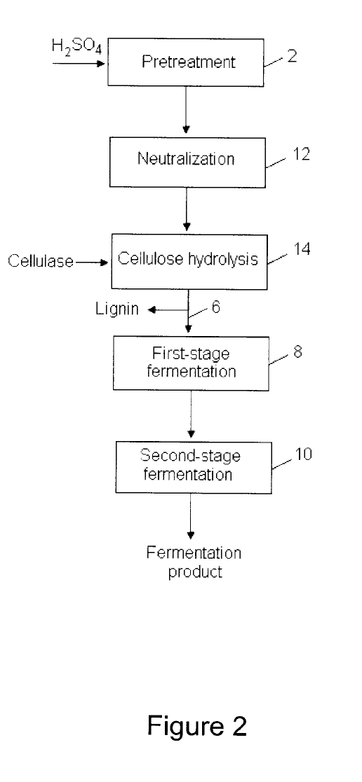

FIGS. 1 and 2 are process flow diagrams depicting the different stages where the sugar stream can be obtained from in a process involving acid pretreatment of a lignocellulosic feedstock to hydrolyze hemicellulose, followed by enzymatic hydrolysis to produce glucose. However, the processes depicted in the figures should not be construed as limiting the invention in any manner and it should be understood that other hydrolysis processes, besides those set forth above, can be used to generate the sugar stream.

Referring now to FIG. 1, there is depicted a process in which a pretreatment 2 is conducted to produce a pretreated feedstock composition, followed by a solids-liquid separation (not shown) of the pretreated feedstock composition to obtain a sugar stream 6 containing sugars arising from the hemicellulose component of the feedstock, i.e., a hemicellulose hydrolyzate. That sugar stream 6 is then sent to a first-stage fermentation 8 which produces a fermented solution enriched in xylose. This, in turn, is followed by a second-stage fermentation 10 to convert the xylose in this stream to a high value product by fermentation. These first- and second-stage fermentations are discussed in more detail below.

The solids portion arising from the solids-liquid separation of the pretreated feedstock composition is subjected to neutralization 12 to achieve a pH between about 4 and about 6 (or higher if alkalophilic cellulases are used) and then fed to cellulose hydrolysis 14 with cellulase enzymes to produce glucose. The lignin may then be removed and burned to supply energy for the plant. That step may be followed by fermentation 16 of glucose resulting from the cellulose hydrolysis to produce a fermentation product. If ethanol is produced during this fermentation, it is concentrated by distillation 18 and this produces a distillate and still bottoms.

An alternative, non-limiting embodiment is depicted in FIG. 2. According to this particular embodiment (in which like reference numbers indicate identical or similar processing steps as FIG. 1), the feedstock is pretreated 2 and then the pretreated feedstock composition is neutralized 12 and subjected to cellulose hydrolysis 14 with cellulase enzymes. After lignin removal, the first 8 and second stage 10 fermentations are carried out, as discussed in more detail below. In this embodiment, the sugar stream fed to the first-stage fermentation 8 contains glucose liberated from the cellulose and thus contains significantly more of this sugar monomer than in the sugar stream sent to the first-stage fermentation in FIG. 1.

It is also considered within the scope of the invention to produce the sugar stream by hydrolyzing the lignocellulosic feedstock in a single step with acid or alkali. This employs harsher conditions to effect hydrolysis of both the hemicellulose and cellulose components of the feedstock. (See, for example, U.S. Pat. No. 5,562,777, which describes acid hydrolysis of cellulose and hemicellulose, the contents of which are herein incorporated by reference). Furthermore, a two-stage acid or alkali hydrolysis is also included within the scope of the invention.

Furthermore, it will be understood that, prior to fermentation, the sugar stream may be subjected to additional processing steps. In one embodiment of the invention, at least a portion of the mineral acid and/or organics acids, including acetic acid, present in the hemicellulose hydrolysate are removed from the sugar stream, for example, by anion exchange. (See, for example, WO 2008/019468, Wahnon et al., which is incorporated herein by reference) Other processing steps that may be conducted prior to the fermentation include concentration by evaporation and/or reverse osmosis.

### Components Present in the Sugar Stream

### Sugars

As discussed previously, hydrolysis of the hemicellulose and cellulose component of a lignocellulosic feedstock yields a sugar stream comprising xylose and glucose. Other sugars that are typically present include galactose, mannose, arabinose, fucose, rhamnose or a combination thereof. Regardless of the means of hydrolyzing the lignocellulosic feedstock (e.g., pretreatment or full acid hydrolysis), the xylose and glucose generally make up a large component of the sugars present in the sugar stream.

If the sugar stream is a hemicellulose hydrolysate resulting from pretreatment, xylose will be the predominant sugar and lesser amounts of glucose will be present, since a modest amount of cellulose hydrolysis typically occurs during pretreatment. According to this embodiment of the invention, the xylose can make up between about 65 and 95 wt % of the combined xylose and glucose content of the sugar stream. According to another embodiment of the invention, the xylose makes up greater than about 50 wt % of the combined xylose and glucose content, between about 65 and about 95 wt %, or between about 70 and about 95 wt % of the combined xylose and glucose content. For example, the xylose may make up greater than 50, 55, 60, 65, 70, 75, 80, 85, 90 or 95 wt % of the combined xylose and glucose content. It will be appreciated by those of skill in the art that the relative amounts of glucose and xylose present in the sugar stream will depend on the feedstock and the pretreatment that is employed.

If the sugar stream results from hydrolysis of the cellulose and hemicellulose components of the feedstock, e.g., full acid or alkali hydrolysis, it will contain all of the sugars listed above, but higher levels of glucose derived from the more complete hydrolysis of the cellulose.

Although the production of a sugar stream comprising xylose and glucose has been described herein, the invention also encompasses conducting the first-stage fermentation on a sugar stream comprising a hexose sugar selected from the group consisting of glucose, galactose, rhamnose, fructose and mannose, and a combination thereof and a pentose sugar selected from the group consisting of xylose, arabinose and fucose, and a combination thereof.

### Inhibitors

In addition to the aforementioned sugars, sugar streams derived from lignocellulosic feedstocks contain a number of compounds that may or may not be inhibitory to the microorganism in the first and second stage fermentations. Furan derivatives such as 2-furaldehyde (furfural) and 5-hydroxymethyl-2-furaldehyde (1-IMF) are inhibitory compounds that originate from the breakdown of the carbohydrate fraction, namely xylose and glucose, respectively, although other inhibitory compounds can be present in sugar streams as set forth below. These compounds can be degraded further by pretreatment or hydrolysis into organic acids including acetic acid, as well as formic, and levulinic acids that are also inhibitory. Additional organic acids found in the sugar stream that may be inhibitory to yeast or other microorganisms include galacturonic acid, lactic acid, glucuronic acid, 4-O-methyl-D-glucuronic acid or a combination thereof. Inhibiting phenolic compounds are also produced by the degradation of lignin, which include vanillin, syringaldeyhde, and hydroxybenzylaldehyde. In particular, vanillin and syringaldehyde are produced via the degradation syringyl propane units and guaiacylpropane units of lignin (Jonsson et al., 1998, Appl. Microbiol. Biotechnol. 49:691).

As discussed previously, acetic acid is a component of sugar streams produced from lignocellulosic material that is highly inhibitory to yeast. The acetate arises from acetyl groups attached to xylan and lignin that are liberated as acetic acid and/or acetate by exposure to acid or other chemicals that hydrolyze the feedstock. (Abbott et al., 2007, FEMS Yeast Res. 7:819-833; Hu et al. 2009, Bioresource Technology 100:4843-4847; Taherzadeh et al., 1997, Chem Eng Sci, 52:2653-5659.). Acetic acid has a pKₐ of about 4.75 (Kₐ of 1.78×10⁻⁵) so that at pH 4.0, about 14.8 mole % of the acid is present as acetate. Thus, the species present in the sugar stream will depend on the pH of the solution. Although it should be appreciated that the practice of the invention is not limited by the pH of the sugar stream, the fermentation is typically conducted at a pH at which acetate is the dominant species in solution. However, the term “acetate” as used herein encompasses acetic acid species. Acetate may be present in the sugar stream at a concentration of between about 0.1 and about 50 g/L, about 0.1 and about 20 g/L, about 0.5 and about 20 g/L or about 1.0 and about 15 g/L. For example, the acetate may be present in the sugar stream at a concentration of about 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 25.0, 30.0, 35.0, 40.0, 45.0 or 50.0 g/L.

The inhibitory compounds set forth above are representative of the compounds present in a sugar stream produced from a lignocellulosic feedstock. A more extensive list of compounds that are present after pretreatment is provided in Klinke et al. (Appl Microbiol Biotechnol, 2004, 66:10-26, the contents of which are incorporated herein by reference). It will be appreciated that the substances present depend on both the raw material and the pretreatment that is employed.

### Processing of the Sugar Stream

### First-Stage Fermentation

According to the invention, the sugar stream is fed to a fermentation reactor(s) of the first-stage fermentation at a dilution rate that enables the microorganism to convert the hexose sugar and optionally also the acetate, in the sugar stream to cell mass. A suitable dilution rate will depend on the microorganism utilized, but can be determined with ease by one of ordinary skill in the art provided with the detailed guidance set forth in Example 1. As a person of ordinary skill in the art would be aware, the term “dilution rate” refers to the ratio of the feed rate to the volume of culture in the vessel. Furthermore, those of ordinary skill in the art will be able to distinguish between a CSTR and a fed-batch dilution rate. That is, in a CSTR configuration, the volume of the culture remains constant and, as such, so does the feed rate. In fed-batch operation, the volume of the culture will continuously increase; thus, the feed rate must also increase in such a manner as to ensure the ratio of the two remains constant.

In practice, determination of a suitable dilution rate involves conducting continuous fermentations at varying dilution rates and under aerobic conditions and then selecting a feed rate at which the glucose and the acetate is consumed by the microorganism and converted to cell mass, while leaving all or a majority of the xylose unconverted. Typically, this involves choosing an initial set point for the feed rate and then feeding the sugar stream to the fermentor at that rate until the concentration of all the components in the effluent stream (i.e., the stream leaving the reactor) stabilizes. If the initial set point does not result in glucose and acetate conversion to cell mass, with little or no xylose consumption, then the process is repeated until the dilution rate achieves this.

According to one embodiment of the invention, the dilution rate is between about 0.01 and about 2.0 h⁻¹, or about 0.01 and about 0.55 h⁻¹, or between about 0.01 and about 0.50 h⁻¹, or between about 0.05 and about 0.45 h⁻¹, or between about 0.1 and about 0.45 h⁻¹, or between about 0.15 and about 0.40 h⁻¹ or between about 0.20 and about 0.40 h⁻¹, or between about 0.30 and about 0.40, or any range therebetween. For example, the dilution rate may be 0.01, 0.05, 0.1, 0.15, 0.20, 0.25, 0.30, 0.35, 0.40, 0.45, 0.50, 0.55, 0.60, 0.70, 0.80, 0.90, 1.0, 1.1, 1.2, 1.3, 1.4, 1.5, 1.6, 1.7, 1.8, 1.9 or 2.0 h⁻¹.

According to another embodiment of the invention, when yeast are employed, the dilution rate is between about 0.01 and about 0.6 h⁻¹, or between about 0.05 and about 0.45 h⁻¹, or between about 0.1 and about 0.45 h⁻¹, or between about 0.15 and about 0.40 h⁻¹ or between about 0.20 and about 0.40 h⁻¹, or between about 0.30 and about 0.40 h⁻¹, or any range therebetween.

The level of glucose and optionally acetate conversion to cell mass and xylose consumption is determined by any suitable quantitative analysis technique known to those of skill in the art. For example, a sample of the effluent from the fermentor may be taken, followed by centrifugation of the cells. The supernatant may be analyzed, for example by HPLC, for the concentration of glucose and xylose. The cell mass may be determined by a standard dry weight technique known to those of skill in the art.

As used herein, the term “aerobic conditions” refers to a fermentation in which air or an oxygen-containing mixture is supplied to the fermentor with a sufficient oxygen transfer rate so as to deliver oxygen to the culture in an amount to ensure that all or a major portion of carbon from glucose and acetate, but little or no xylose, is dissimilated to carbon dioxide (CO₂) and assimilated into cell mass. The O₂ consumption rate is microorganism dependent, but can be determined with ease by those of ordinary skill in the art.

A suitable method for ensuring that aerobic conditions are maintained during the fermentation (i.e., that a culture is respiring) is to measure the amount of oxygen consumption and the carbon dioxide production and then calculate a respiratory quotient (RQ), which is the ratio of CO, produced to O₂ consumed. In one embodiment of the invention, oxygen or an oxygen-containing mixture (including, but not limited to, air) is supplied to the fermentor at a rate that achieves an RQ of about 0.9 to about 1.1. (At an RQ of 1, a culture is said to be “fully respiring”). In another embodiment of the invention, the fermentation is carried out at an RQ between about 0.92 to about 1.08, more preferably between 0.95 and 1.05, or at an RQ of about 0.9, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09 or 1.1. In yet another embodiment of the invention, the RQ is about 1.

As discussed previously, the fermented solution from the first-stage fermentation is enriched in the xylose relative to the sugar stream fed to fermentation reactor. By this it is meant that, by virtue of the conversion of the glucose to cell mass, the ratio of the xylose relative to all other sugars in solution (wt:wt) is greater in the fermented solution than the sugar stream fed to the fermentor. The xylose:glucose (wt:wt) can be at least 20:1, 30:1, 40:1, 50:1, 60:1, 70:1, 80:1, 90:1, 100:1, 200:1, 300:1, 400:1, 500:1, 600:1, 700:1, 800:1, 900:1, 1000:1, 10,000:1, 100,000:1 or higher. The concentration of the xylose after the fermentation may be below the detection limit of the instrument employed to measure its concentration.

It is preferable that the microorganism converts as much of the glucose as possible to cell mass within practical limits, although low concentrations may be present after the fermentation. Optionally, acetate is also fermented to cell mass by the microorganism. Depending on the microorganism's metabolism of xylose in the presence of glucose, it may be that residual amounts of glucose remain, while at the same time residual amounts of xylose are consumed at the operational dilution rate. In other words, the conditions of an ideal dilution rate set forth in Example 1 may not be practically achievable.

In another embodiment of the invention, the fermentation is carried out at a temperature that is about 20° C. to about 50° C. or any value therebetween. For example, the temperature may be from about 25° C. to about 40° C., or at a temperature of about 25° C. to about 35° C., or at a temperature of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50° C.

In another embodiment, the pH is from about 2 to about 7, or any value therebetween, for example at a pH from about 2.5 to 6.5, a pH from about 3.5 to 6.5, or at a pH of 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8 or 7.0. To maintain the desired pH range of from about 2 to about 7, the fermentation may need continuous adjustment to this pH range. Those of skill in the art will be aware of the chemicals suitable for adjusting the pH of the fermentation and non-limiting examples of suitable chemicals include ammonium hydroxide, sodium hydroxide, potassium hydroxide, sulfuric acid, phosphoric acid and hydrochloric acid.

The sugar stream may also be supplemented with additional nutrients required for growth of the fermentation microorganism. For example, when yeast are utilized, yeast extract, specific amino acids, phosphate, nitrogen sources, salts, trace elements and vitamins may be added to the sugar stream to support growth and optimize its productivity.

The first-stage fermentation may be conducted in continuous or fed-batch modes, although continuous processes are preferred. Preferably, the fermentation reactors are agitated with mixing. The fermentation may be conducted using a series of reactors or in one reactor.

Suitable microorganisms for use in the fermentation can be any one of a number of bacterial or fungal strains that are capable of consuming glucose over xylose. Examples of fungal organisms include yeast and filamentous fungi. Preferred yeast includes strains of the genus Saccharomyces, for example, S. cerevisiae; strains of the genus Pichia, for example, P. stipitis or P. pastoris; strains of the genus Candida, for example. C. tropicalis, C. guilliermondii, C. utilis, C. arabinofermentans, C. diddensii, C. sonorensis, C. shehatae, C. boidinii and C. paripsilosis; strains of the genus Hansenula, for example, H. anomala and H. polymorpha; strains of the genus Kluyveromyces, for example, K. marxianus and K. fragilis; and strains of the genus Schizosaccharomyces, for example S. pombe. Filamentous fungi that are contemplated for use in the present invention include Aspergillus, Humicola and Trichoderma.

By “preferentially ferments the glucose over the xylose”, it is meant that the microorganism can assimilate the glucose into cell mass preferentially over the xylose. In order to determine the preferential consumption of glucose over xylose, one would grow the microorganism in question on media containing equal concentrations of the glucose and xylose and monitor their consumption rates.

Preferable bacterial organisms include strains of Escherichia, in particular E. coli; strains of the genus Zymomonas, for example Z. mobilis; strains of the genus Zymobacter, for example, Z. palmae; strains of the genus Klebsiella, for example, K. oxytoca; strains of the genus Leuconostoc, for example, L. mesenteroides; strains of the genus Clostridium, for example, C. phytofermentans; strains of the genus Enterobacter, for example, E. aerogenes; strains of the genus Thermoanaerobacter, for example T. BG1L1; strains of the genus Thermoanaerobacter, for example, T. ethanolicus, T. thermosaccharlyticum and T. mathranii; as well as strains of the genus Lactobacillus.

Particularly preferred microorganisms include those of a genus selected from Saccharomyces, Pichia, Candida and Hansenula, for example, but not limited to, those species set forth above.

The microorganism used in the first-stage fermentation may also be capable of converting other inhibitors besides acetate to their respective less inhibitory analogs. For example, furfural may be converted to furoic acid or furfural alcohol by the microorganism. It is known that in fermentations with Saccharomyces cerevisiae under anaerobic conditions, furfural is converted to furfural alcohol, while under aerobic conditions, furoic acid is produced. (Horvath et al., 2003. Applied and Environmental Microbiology, 69(7):4076-4086). The inhibitor, 5-hydroxymethylfurfural, may also converted to a less inhibitory analog by the microorganism in the first-stage fermentation. (See Liu and Moon, 2009, Gene 446:1-10).

The conversion of glucose to cell mass may be accompanied by the expression of one or more active proteins of interest. For example, this may involve the overexpression of one or more recombinant proteins by known techniques such as by increasing the copy number of the gene encoding the protein of interest or by increasing the binding strength of transcription factors to the promoter region of the gene. The number of copies of the gene may be increased by introducing multiple copies of an expression vector without genomic integration or by transforming a host cell with DNA encoding a protein of interest so that multiple copies are integrated into the genome by recombination. Deletion strains may also be produced in order to vary the relative amounts of one protein expressed with respect to another. The protein that is expressed may be heterologous or endogenous.

Advantageously, in one embodiment of the invention, if xylan remains in the sugar stream fed to the first-stage fermentation, the protein expressed may be a xylanase, which in turn, can hydrolyze the xylan, thereby increasing the concentration of xylose fed to the second-stage processing.

After the fermentation is complete, the microorganisms in the fermented solution can be separated from the solution and then the supernatant, which is enriched in the xylose, can be sold as a commercial product, typically after purification. For example, if the fermented solution contains xylose, it may be crystallized as disclosed in U.S. Pat. No. 3,784,408 after removal of spent cells. If a protein(s) is expressed, it may be isolated from the fermented solution using conventional techniques. Subsequently, the fermented solution may be subjected to a second-stage processing step to convert the xylose to another chemical, as discussed in more detail below.

### Second-Stage Fermentation or Non-Biological Conversion

The stream comprising xylose produced in the first-stage fermentation can be used to produce a variety of fermentation products, examples of which include alcohols, including ethanol and butanol; sugar acids including xylonic acid and arabonic acid; sugar alcohols including xylitol, arbitol, erythritol, galactitol and mannitol; organic acids including citric acid, malic acid, succinic acid, pyruvic acid, acetic acid, itaconoic acid and lactic acid; ketones including acetone; amino acids, including glutamic acid; gases including H₂ and CO₂, antibiotics including penicillin and tetracycline; enzymes; hormones; and vitamins.

Although the sugar acids, organic acids and amino acids listed above are denoted in their acid forms, the species present will depend on the pH of the solution. It should be understood that the invention is not limited by the pH of the fermented solution, or any stream derived therefrom, or the particular species that is dominant in solution at any particular pH.

In a preferred embodiment, the xylose is converted to a sugar alcohol. The sugar alcohol may be selected from xylitol, arbitol, erythritol, mannitol and galactitol. Preferably, the sugar alcohol is xylitol. In one embodiment of the invention, the xylose is fermented to xylitol by yeast. Yeast that are capable of converting xylose to xylitol include strains of Candida such as strains of C. tropicalis, C. guilliermondii, C. polymorpha, C. boidinii, C. intermedia, C. mogii, C. parapsilosis, C. pseudotropicalis, C. shehatae and C. utilis; strains of Pichia, such as P. stipitis, P. segobiensis and P. anomala; strains of Pachysolen such as P. tannophilus, strains of Hansenula such as H. anomala, strains of Debaryomyces such as D. hansenii, strains of Kluyveromyces such as K. fragilis and K. marxianus, strains of Saccharomyces cerevisiae and strains of Schizosaccharomyces pombe. According to one embodiment of the invention, the yeast strain is Candida, preferably C. tropicalis. Bacteria are also known to produce xylitol, including Corynebacterium sp., Enterobacter liquefaciens and Mycobacterium smegmatis. The invention also encompasses genetically modified yeast or bacteria that are capable of converting xylose to xylitol. (See Kim et al., 1999, Journal of Biotechnology, 67:159-171 and Lee et al., 2003, Applied and Environmental Microbiology, 69(10):6179-6188).

After removal of the yeast or bacteria cells, the xylitol in the fermented solution may be purified by a chromatographic separation. Typically, this is conducted with a strongly acidic cation exchange resin, preferably with a preliminary de-salting step. Subsequently, the xylitol may be crystallized using conventional crystallization techniques.

In another preferred embodiment, the xylose is converted to a sugar acid, including, but not limited to, xylonic acid.

According to another embodiment of the invention, the xylose is converted to an alcohol by fermentation with a naturally-occurring or recombinant bacterium or fungus. Preferably, the fungus is a yeast strain, although filamentous fungi are also considered within the scope of the invention. The alcohol may be ethanol or butanol, preferably ethanol. The xylose may be fermented to ethanol by a yeast strain that is naturally capable of converting xylose to ethanol or that has been genetically modified so that it is capable of producing this valuable byproduct. For example, Saccharomyces cerevisiae strains that are not naturally capable of converting xylose to ethanol can be genetically modified to do so by the introduction of xylose reductase, xylitol dehydrogenase and xylulokinasc (see U.S. Pat. No. 5,789,210 (Ho et al.), which is incorporated herein by reference). Furthermore, Saccharomyces cerevisiae strains that possess the ability to convert xylose to ethanol can be isolated in the laboratory by non-recombinant methods. For example, such yeasts may be obtained by the mating of genetically diverse strains and selecting mutants that grow on xylose as a sole carbon source. (See US 2008/0009047, Bell et al.) US 2008/0261287 (incorporated herein by reference) discloses genetic modifications of eukaryotic cells that have been transformed to express xylose isomerase (XI). The introduction of XI confers to the host cell the ability to isomerize xylose to xylulose, thereby increasing the production of a fermentation product such as ethanol. WO 2008/130603 (Abbas et al., which is incorporated herein by reference) discloses Hansenula polymorpha strains with increased production of ethanol from xylose. Other yeast strains that may be used in accordance with the invention include Pichia stipitis and Candida shehatae mutants isolated by the method disclosed in U.S. Pat. No. 5,126,266 (Jeffries et al., which is incorporated herein by reference). In addition, a recombinant Zymomonas mobilis strain has been described that is capable of utilizing xylose to produce ethanol by a genetic modification that reduces glucose-fructose oxidoreductase activity (see WO 2008/133638, incorporated herein by reference). Further examples of microorganisms that can convert xylose to ethanol, include, but are not limited to, Escherichia coli, Thermoanaerobacter mathranii, Escherichia coli, Thermoanaerobacter mathranii and Clostridium. phytofermentans.

The ethanol may then be recovered from the fermentation broth by distillation. After distillation, further removal of water may be carried out using molecular sieves or other expedients.

If the same microorganism is used in the second-stage fermentation as the first-stage then it is typically advantageous to send the fermented solution from the first fermentation containing the microorganism to the second-stage fermentation without removing the microorganisms. This is especially beneficial as it can reduce the requirements for recycle of the microorganisms in the second-stage fermentation. However, it is also possible to use a different microorganism in the second-stage fermentation, in which case the cells in the fermented solution are separated from the fermented solution, for example by centrifugation, filtration and similar expedients, and the filtrate then sent to the second-stage fermentation for fermentation by another microorganism.

The second-stage fermentation may be supplemented with additional nutrients required for growth of the fermentation microorganism. For example, if yeast are employed in the fermentation, yeast extract, specific amino acids, phosphate, nitrogen sources, salts, trace elements and vitamins may be added to the fermentor to support the growth and optimize the productivity of the yeast.

The second-stage fermentation may be conducted in batch, continuous or fed-batch modes, with or without agitation. Preferably, the fermentation reactors are agitated lightly with mixing. A typical, commercial-scale fermentation is conducted using a series of reactors, such as 1 to 6, or any number therebetween.

Chemical means for producing a commercial product from the xylose (i.e., non-biological processes) are also considered within the scope of the present invention since it may be beneficial to remove sugar impurities and inhibitors prior to the chemical conversion of the xylose. For example, xylose remaining after the first-stage fermentation can be converted to xylitol by catalytic hydrogenation using a nickel catalyst (e.g. a Raney catalyst), and such processes are known to proceed more efficiently in the absence of, or with reduced concentrations of, unwanted sugars and other impurities.

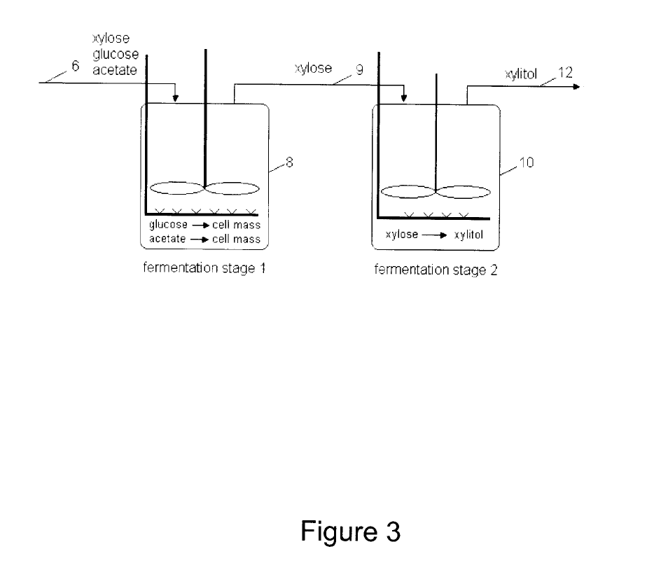

Referring now to FIG. 3, there is shown a non-limiting example of the two-stage fermentation conducted in accordance with embodiments of the invention. According to this non-limiting example, a sugar stream 6, for example a hemicellulose hydrolyzate, comprising at least xylose, glucose and acetate is fed to the first-stage fermentation 8. During this first-stage fermentation 8, the glucose and acetate are converted to cell mass using Candida tropicalis. The dilution rate is determined as set forth in Example 1 and the fermentation is conducted under aerobic conditions so as to achieve an RQ of about 1. This produces a stream 20 that is enriched for xylose relative to the feed stream, i.e., it contains reduced concentrations of acetate and glucose. The xylose-enriched stream 9 and the yeast generated in the first-stage fermentation are then fed to a second-stage fermentation 10 that converts xylose to xylitol. Thus, unwanted components, namely glucose and acetate in this particular embodiment, are converted to cell mass for the second fermentation. This is particularly advantageous since it eliminates or reduces the requirements for yeast recycle in the second fermentation. Furthermore, the inhibitory effect of acetate in the second fermentation is reduced or eliminated. In this particular embodiment, the second-stage fermentation 10 is also conducted under aerobic conditions. The fermented solution 12 comprising xylitol is then subjected to centrifugation to remove the spent yeast cells and the filtrate is then processed to recover xylitol.

### EXAMPLES

### Example 1

### Determination of a Dilution Rate to Convert Hexose Sugar(s) and Acetate to Cell Mass

The following example describes a method for determining a dilution rate suitable for converting hexose(s) and acetate in the sugar stream to cell mass without xylose consumption.

Firstly, a sufficient amount of air is continuously supplied with a mixing rate to ensure that enough oxygen is transferred to maintain a fully respiring culture. Otherwise, the hexose sugar(s) consumed will be fermented and form products such as ethanol or lactic acid rather than cell mass.

To determine the operational dilution rate (D_{Op}), a series of continuous-stirred tank reactor (CSTR) steady-state fermentations are conducted at varying dilution rates and are analyzed for the concentration of sugars exiting the fermentor; the stream exiting the fermentor is referred to as the effluent. Initially, a fermentor is prepared with the appropriate media to grow up an initial starter culture. Once the starter culture has been grown up, the CSTR phase of the experiment commences. The CSTR may be set to operate at a constant working volume. The sugar stream containing xylose and glucose sugars and acetate is then continuously feed to CSTR fermentor with simultaneous removal of effluent at the same rate the feed is entering the fermentor. The ratio of the feed rate to the volume of culture in the vessel is the dilution rate. For example, at a volumetric feed rate of 1 L/h, a working volume of 2 L would result in a dilution rate of 1 L/h/2 L=0.5 h⁻¹. An initial set point for the feed rate is chosen, and the fermentor is fed at this rate until the concentrations of all the components in the effluent stream stabilizes, i.e. the system reaches steady state. One of five scenarios may be true at the selected dilution rate:

Low dilution rate—both glucose and acetate and xylose completely consumed.

Moderately low dilution rate—all glucose and acetate consumed and partial consumption of xylose.

Ideal dilution rate—all glucose and acetate consumed, no xylose consumed.

Moderately high dilution rate—partial glucose and acetate consumption, no xylose consumption.

High dilution rate—no glucose and acetate or xylose consumption (cells washout).

Based on which of the five scenarios are initially encountered, the initial dilution rate may or may not need to be increased or decreased. If the ideal dilution rate is not realized initially, then the feed rate will be altered and the CSTR will again be allowed to achieve steady-state. Incremental changes in dilution rate can be made until the ideal dilution rate is achieved. Using Monod kinetics, a knowledge of growth parameters such as the maximum growth rate (μₘ) and critical growth rate (μ_{critical}) for glucose and xylose, as well as affinity constants for glucose and xylose will help to narrow down the target dilution rate prior to commencing the experiment.

For fermentation of the same microorganism, the dilution rate to achieve the xylose-enriched stream will be the same, whether fed-batch or CSTR. Thus, CSTR experiments would preferentially be performed to identify D_{Op} regardless of the desire to run in either fed-batch or continuous modes. Once the dilution rate has been identified via CSTR experiments, the dilution rate will be tested in fed-batch mode to determine if indeed the dilution rate is appropriate. Because no effluent is removed from the fed-batch fermentor, the volume in fermentor is continuously increasing; thus, the feed rate must also be changing at such a rate that the ratio of the feed rate to the volume remains constant.

### Example 2

### Respirative Continuous Detoxification of Inhibitors and Removal of Glucose and Acetate in Pretreated Lignocellulosic Feed Stock Using Candida tropicalis with Subsequent Fermentation of Effluent to Xylitol

### (a) Feed Preparation.

Hydrolyzate from a dilute acid pretreated lignocellulosic biomass conducted as set forth in U.S. Pat. No. 4,461,648 (incorporated herein by reference) was concentrated by evaporation. It contained 75 g/L xylose, 10 g/L glucose, and 2.5 g/L acetic acid.

The following salts, trace elements and vitamins were added, on a per liter basis, to the feed for the first-stage fermentation.

TABLE 1
Salts, trace compounds and vitamins
added to the first-stage fermentation

| | | Concentration |
|---|---|---|
| Constituent | Species present | of species (g/L) |
| Salts | (NH₄)₂SO₄ | 5 |
| | K₂HPO₄ | 3 |
| | MgSO₄•7H₂O | 0.5 |
| Trace | ZnSO₄•7H₂O | 0.0045 |
| compounds | MnCl₂•4H₂O | 0.001 |
| | CoCl₂•6H₂O | 0.0003 |
| | CuSO₄•5H₂O | 0.0003 |
| | Disodium molybdate dihydrate | 0.0004 |
| | CaCl₂•2H₂O | 0.0045 |
| | FeSO₄•7H₂O | 0.003 |
| | Boric acid | 0.001 |
| | KI | 0.0001 |
| Vitamins | D(−)biotin | 5 × 10⁻⁵ |
| | Calcium D(+)pantothenate | 0.001 |
| | Nicotinic acid | 0.001 |
| | Myo-inositol | 0.001 |
| | Thiamine hydrochloride | 0.001 |
| | Pyridoxol hydrochloride | 0.001 |
| | p-amino benzoic acid | 0.002 |

The above trace compounds and vitamins were added from a stock solution containing all the components set forth above. Such stock solutions were prepared according to Verduyn et al., 1992, Yeast 8(7):501-170, which is incorporated herein by reference.

Subsequently, 10 L of this feed was transferred to a sterile 14 L fermentor for the initial batch phase of the CSTR.

### (b) Inoculum Preparation.

10⁹ cells of C. tropicalis (ATCC 1369) was used to inoculate a 2 L baffled flask containing 1000 mL of media containing the aforementioned salts, vitamins, and trace compounds in the concentrations previously mentioned along with 60 g/L glucose. Cells were cultivated for 48 h in a shaker incubator at 30° C. and 160 rpm. The culture was centrifuged at 3000×g for 5 min. The cells were re-suspended in 20 mL of media and transferred to a flask containing a medium identical in composition as that described in the feed preparation. The culture was incubated for 24 h at the aforementioned conditions. After cultivation, the cells were centrifuged a second time using the same conditions and all supernatant but 20 mL was decanted. The cells were then re-suspended and transferred aseptically to inoculate the 14 L fermentor described previously.

### (c) Initial Batch Start Up of CSTR.

Cells were grown for 24 h in batch culture in a New Brunswick Scientific 14 L fermentor in a working volume of 10 L at 30° C., controlled at pH 5 using 10% ammonium hydroxide, an agitation rate of 800 rpm, and an air flow rate of 12 standard liters per minute. Once the pH of the culture began to increase, the feed and harvest pumps were activated.

### (d) First Stage Processing: CSTR OPERATION.

A working volume of 10 L was targeted with a dilution rate of 0.3 h⁻¹. At this dilution rate, residual glucose was not detected (≦0.1 g/L) and the residual xylose concentration was 45 g/L at steady-state resulting in a ratio of 450:1 (xylose:glucose). At steady-state, four samples were taken at regular intervals over a period of one turn-over of the vessel volume (3.3 h). The samples were analyzed for cell mass using dry cell weight (Rice et al., 1980, Am. Soc. Brew. Chem. Journal 38:142-145, which is incorporated herein by reference). An Agilent 1100 Series HPLC system equipped with a Biorad Micro Guard Refill Cartridge and a Varian METACARB 87H column was used to determine the glucose, xylose, xylitol, ethanol, glycerol, lactate and acetate concentrations of each sample. The eluant used for the HPLC analysis was 5 mM aqueous sulfuric acid. The HPLC was operated at 0.6 mL/min using an isocratic pump and the column was held constant at 50° C. The unit was equipped with an Agilent 1100 Series RI detector and 1200 Series variable wavelength detector operated at 210 nm.

The results of the sample analysis are presented in Table 2.

### Example 3

### Respirative Continuous Detoxification of Inhibitors and Removal of Glucose and Acetate in Pretreated Lignocellulosic Feedstock Using Superstart™ (Saccharomyces cerevisiae)

An identical procedure as that described in Example 2 (parts a through d) was employed except that the species of yeast utilized was Superstart™, a commercial Saccharomyces cerevisiae strain.

Table 2 summarizes the results from the CSTR experiments performed with C. tropicalis (ATCC 1369) and Superstart™.

TABLE 2
Results from the aerobic CSTR experiments performed with
C. tropicalis (ATCC 1369) and Superstart ™.

| | Dilution | Glucose | Xylose | Acetic | Glucose | Xylose | Acetic | Cells |
|---|---|---|---|---|---|---|---|---|
| | Rate | Fed | Fed | Acid Fed | Out | Out | Acid Out | mass |
| Microorganism | (h⁻¹) | (g/L) | (g/L) | (g/L) | (g/L) | (g/L) | (g/L) | (g/L) |
| C. tropicalis | 0.3 | 10 | 75 | 4.5 | 0 | 45 | 0 | 13 |
| Superstart ™ | 0.15 | 10 | 100 | 4.5 | 0 | 100 | 0 | 3 |

### Example 4

### Respirative Continuous Detoxification of Inhibitors and Removal of Glucose and Acetate in Pretreated Lignocellulosic Feedstock Using Y108-1 LNH-ST

Aerobic CSTR experiments were performed with a recombinant Saccharomyces strain referred to herein as “Y108-1 LNH-ST” containing xylose reductase (XR) and xylitol dehydrogenease (XDH) genes from Pichia stipitis (as described in co-pending and co-owned U.S. Ser. No. 61/307,536).

A similar procedure as that described in Example 2 (parts a through d) was carried out with some exceptions. The process sugar hydrolyzate stream contained 115 g/L xylose and 10 g/L glucose and was filter-sterilized through a 0.2 μm membrane prior to its introduction to the first-stage fermentation. Also, during inoculum preparation, after the cells were centrifuged and transferred to a flask containing process sugars supplemented with media, the culture was incubated for 72 hours rather than 24 hours.

During the first stage CSTR operation, feeding was started at 49 hours and the five turnovers necessary to achieve steady-state was achieved at 221 hours. The CSTR was run at a dilution rate of 0.0306 h⁻¹, which was based on a measured working volume of 8.55 L and feed rate of 0.262 L/h. An initial batch start-up was conducted as described previously. Data from 221 and 242 hours represents data used for steady-state.

TABLE 3
Results from the aerobic CSTR experiments performed
with Y108-1 LNHST (feed started at 49 hours).

| | | | | | acetic | lactic | | cell | Y |
|---|---|---|---|---|---|---|---|---|---|
| time | xylose | glucose | xylitol | glycerol | acid | acid | ethanol | mass | g cell mass/(g |
| (h) | (g/L) | (g/L) | (g/L) | (g/L) | (g/L) | (g/L) | (g/L) | (g/L) | glucose + xylose) |
| 0 | 109 | 17.1 | 0 | 0 | 0.523 | 0 | 1.02 | 0.74 | |
| 46.2 | 0 | 0 | 10.6 | 3.67 | 0 | 0 | 0.30 | 37.7 | 0.293 |
| 98.5 | 50.2 | 0 | 0 | 0 | 0 | 0 | 1.22 | 27.5 | 0.362 |
| 121 | 59.8 | 0 | 0 | 0 | 0 | 0 | 0 | 24.0 | 0.362 |
| 147 | 63.4 | 0 | 0 | 0 | 0 | 0 | 0 | 20.0 | 0.319 |
| 171 | 58.4 | 0 | 0 | 0 | 0 | 0 | 0 | 18.5 | 0.274 |
| 192 | 56.0 | 0 | 0 | 0 | 0 | 0 | 0 | 20.5 | 0.292 |
| 221 | 44.7 | 0 | 0 | 0 | 0 | 0 | 0 | 24.5 | 0.301 |
| 242 | 45.1 | 0 | 0 | 0 | 0 | 0 | 0 | 24.8 | 0.306 |

The results in Table 3 demonstrate that during the CSTR phase (after 49 hours), all glucose and acetate are converted to cells and the effluent is enriched in xylose (xylose:glucose>>20:1).

1. A method for producing a fermentation product from xylose, said method comprising:
(i) obtaining a sugar stream comprising at least glucose and xylose, said sugar stream resulting from the hydrolysis of a lignocellulosic feedstock;
(ii) reducing the concentration of glucose in the sugar stream by fermenting said sugar stream in a first-stage fermentation with a yeast that preferentially ferments the glucose over the xylose, said fermenting being conducted under aerobic conditions and comprising continuously feeding the sugar stream to a fermentation reactor at a dilution rate that allows the yeast to produce cell mass from the glucose preferentially over the xylose so as to produce a fermented solution that is enriched in the xylose relative to said sugar stream; and
(iii) subsequent to step (ii), fermenting the xylose remaining after the first-stage fermentation with yeast to produce the fermentation product.

2. The method of claim 1, wherein the ratio of the amounts of xylose:glucose (wt:wt) in the fermented solution produced in the first-stage fermentation is at least 20:1.

3. The method of claim 1, wherein the sugar stream further comprises acetate and wherein the acetate is converted to cell mass by the yeast in the first-stage fermentation.

4. The method of claim 1, wherein the yeast in the first and second stage fermentations is identical.

5. The method of claim 1, wherein the first-stage fermentation is a continuous process or a fed-batch process.

6. The method of claim 1, wherein the sugar stream resulting from the hydrolysis of the lignocellulosic feedstock further comprises furfural and wherein the furfural is converted to furoic acid or furfural alcohol by the yeast in the first-stage fermentation.

7. The method of claim 1, wherein the sugar stream resulting from the hydrolysis of the lignocellulosic feedstock further comprises 5-hydroxymethylfurfural and wherein the 5-hydroxymethylfurfural is converted to a less inhibitory analog by the yeast in the first-stage fermentation.

8. The method of any claim 1, wherein the xylose makes up at least about 65 wt % of the combined glucose and xylose content of the sugar stream.

9. The method of any claim 1, wherein the yeast in the first-stage fermentation and the second-stage fermentation is a yeast strain that ferments the xylose to xylitol.

10. The method of claim 9, wherein the yeast in the first-stage fermentation and the second-stage fermentation belongs to a genus selected from the group consisting of Candida, Pichia, Pachysolen, Hansenula, Debaryomyces, Kluyveromyces and Schizosaccharomyces.

11. A method for producing xylitol from xylose, said method comprising:
(i) obtaining a sugar stream comprising at least glucose and xylose, said sugar stream resulting from the chemical pretreatment of a lignocellulosic feedstock, wherein the sugar stream contains at least 65% xylose relative to the combined content of glucose and xylose present in said sugar stream;
(ii) reducing the concentration of glucose and acetate in the sugar stream by fermenting said sugar stream in a first-stage fermentation with a Candida, Pichia, Pachysolen, Hansenula, Debaryomyces, Kluyveromyces or Schizosaccharomyces strain that preferentially ferments the glucose over the xylose, said fermenting being conducted under aerobic conditions and comprising continuously feeding the sugar stream to a fermentation reactor at a dilution rate that allows the yeast to produce cell mass from the glucose and acetate preferentially over the xylose so as to produce a fermented solution that is enriched in the xylose relative to said sugar stream; and
(iii) subsequent to step (ii), fermenting the xylose in the fermented solution with the Candida, Pichia, Pachysolen, Hansenula, Debaryomyces, Kluyveromyces or Schizosaccharomyces strain to produce the xylitol.

12. A method for producing a fermentation product from xylose, said method comprising:
(i) obtaining a sugar stream comprising at least glucose and xylose, said sugar stream resulting from the hydrolysis of a lignocellulosic feedstock;
(ii) reducing the concentration of glucose in the sugar stream by fermenting said sugar stream in a first-stage fermentation with a microorganism that preferentially ferments the glucose over the xylose, said fermenting being conducted under aerobic conditions and comprising continuously feeding the sugar stream to a fermentation reactor at a dilution rate that allows the yeast to produce cell mass from the glucose preferentially over the xylose so as to produce a fermented solution that is enriched in the xylose relative to said sugar stream; and
(iii) subsequent to step (ii), fermenting the xylose remaining after the first-stage fermentation with a microorganism to produce the fermentation product.

13. A method for producing a fermentation product from a pentose sugar, said method comprising:
(i) obtaining a sugar stream comprising at least a hexose sugar and a pentose sugar, said sugar stream resulting from the hydrolysis of a lignocellulosic feedstock;
(ii) fermenting said sugar stream resulting from the hydrolysis of the lignocellulosic feedstock with a microorganism that preferentially ferments the hexose sugar over the pentose sugar, said hexose sugar selected from the group consisting of glucose, galactose, rhamnose, fructose and mannose, and combinations thereof, and said pentose sugar selected from the group consisting of xylose, arabinose and fucose, and combinations thereof, said fermenting being conducted under aerobic conditions and comprising continuously feeding the sugar stream to a fermentation reactor at a dilution rate that converts the hexose sugar to cell mass preferentially over the pentose sugar, thereby reducing the concentration of the hexose sugar in the sugar stream so that the fermented solution thus produced is enriched in the pentose sugar relative to said sugar stream; and
(iii) subsequent to step (ii), converting the pentose sugar to a chemical compound.

14. The method of claim 13, wherein the sugar stream resulting from the hydrolysis of the lignocellulosic feedstock further comprises furfural and wherein the furfural is converted to furoic acid or furfural alcohol by the microorganism.

15. The method of claim 13, wherein the sugar stream resulting from the hydrolysis of the lignocellulosic feedstock further comprises 5-hydroxymethylfurfural and wherein the 5-hydroxymethylfurfural is converted to a less inhibitory analog by the microorganism.
